(19) United States
(12) Patent Application Publication
(10) Pub. No.: US 2020/0383635 A1
(43) Pub. Date: Dec. 10, 2020

(54) METHOD, SYSTEM AND NON-TRANSITORY COMPUTER READABLE MEDIUM

(71) Applicant: NEC Corporation, Minato-ku, Tokyo (JP)

(72) Inventors: Ni Ni SOE, BENDEMEER ROAD (SG); Charles CHOY, BENDEMEER ROAD (SG)

(73) Assignee: NEC Corporation, Minato-ku, Tokyo (JP)

(21) Appl. No.: 16/957,952

(22) PCT Filed: Jan. 10, 2019
(86) PCT No.: PCT/JP2019/000497

§ 371 Date: Jun. 25, 2020

(30) Foreign Application Priority Data
Jan. 31, 2018 (SG) 10201800833T

(51) Int. Cl. A61B 5/00; A61B 5/11; A61B 5/0488; G16H 50/30; G16H 20/30

(52) U.S. Cl. CPC A61B 5/4848; A61B 5/7275; A61B 5/11; A61B 2562/0219; G16H 50/30; G16H 20/30; A61B 5/0488

(57) ABSTRACT

A method and system (301) for monitoring and determining progress of a patient's rehabilitative treatment are provided. The system (301) includes two or more sensing devices and a predictive patient recovery potential module. The two or more sensing devices include a first device for sensing physiological performance by the patient while moving a portion of the patient's body and generating a first signal in response thereto and a second device for sensing body portion movement by the patient while moving the portion of the patient's body and generating a second signal in response thereto. The predictive patient recovery potential module is coupled to the two or more sensing devices and includes a categorization module (510) and a functional performance recovery level module (514).

### TECHNICAL FIELD

The present invention generally relates to methods and devices for patient functional ability determination, and more particularly relates to methods and systems for quantitatively determining a patient's significant, latent and manifested functional abilities.

### BACKGROUND ART

In today's world, brain injuries, including brain injuries resulting from cardiovascular disease, are a leading cause of death and a leading increase in a patients' post-injury disability. The ability to live independently after a brain injury depends largely on the patient's recovery of motor function and functional abilities after the brain injury. Therefore, accurate assessment of functional abilities provides substantial assistance for rehabilitation planning and support realistic goal-setting by clinicians, therapists and patients.

In addition, it is important to understand a patient's current functional condition in order to provide suitable treatment strategies. To understand the current stage of a patient's functional abilities, quantitative determination of motor function is a strong clinically relevant indicator of treatment effectiveness. However, current rehabilitation processes are based on subjective scoring of a functional assessment of selected patient movements by a trained clinical therapist. For example, a typical finger extension assessment by a clinical therapist requires a patient to extend their fingers against gravity while the therapist presses down on the fingers using some resistance. The therapist then scores the patient based on their maintenance of the finger extension. In addition to the subjectivity of manual muscle test (MMT) muscle strength scoring, such assessment of motor function and quantification of functional abilities is also flawed because current objective/subjective scoring of muscle strength is based on an ordinal measure of the clinical MMT score and, while four is twice two, it cannot be concluded that a patient with a MMT score of four has twice the muscle strength of a patient with a MMT score of two.

Using motion-sensing to objectively assess motor function and quantify the functional abilities has also been attempted, yet the motion-sensing and subjective/objective scores rarely capture the actual functional performance of a patient. In addition, use of a combination of inertial measurement units (IMUs) and acoustic sensors (for example, bioacoustics sensors such as mechanomyography (MMG) sensors) has been used for sensing muscle activity or monitoring fetal movement but also lack the ability to objectively reflect the actual functional performance of a patient. If only IMU scoring is done, important information on muscle activity is not captured and the IMU score is not patient-relevant. Similarly, if only MMG or electromyography (EMG) scoring of muscle activity is used, important information on the patient's range of motion against gravity or resistance is missing. Some patients may have EMG contraction but limited or no movement and some patients may have movement but limited or weak EMG contractions.

Thus, what is needed is an objective assessment of motor function and quantification of functional abilities which reflects the individual patient's functional abilities. Furthermore, other desirable features and characteristics will become apparent from the subsequent detailed description and the appended claims, taken in conjunction with the accompanying drawings and this background of the disclosure.

### SUMMARY OF INVENTION

### Solution to Problem

According to at least one embodiment of the present invention, a method for monitoring and determining progress of a patient's rehabilitative treatment is provided. The method includes sensing physiological performance and body portion movement while moving a portion of the patient's body, generating a first signal in response to the sensed physiological performance of the portion of the patient's body, and generating a second signal in response to the sensed body portion movement of the portion of the patient's body. The method further includes determining a latent category in response to the first signal, determining a manifested category in response to the second signal, and determining a significant category in response to both the first signal and the second signal. Finally, the method includes determining the patient's rehabilitative treatment progress in response to all of the latent category, the manifested category and the significant category.

According to another embodiment of the present invention, a system for monitoring and determining progress of a patient's rehabilitative treatment is provided. The system includes two or more sensing devices and a predictive patient recovery potential module. The two or more sensing devices include a first device for sensing physiological performance by the patient while moving a portion of the patient's body and generating a first signal in response thereto and a second device for sensing body portion movement by the patient while moving the portion of the patient's body and generating a second signal in response thereto. The predictive patient recovery potential module is coupled to the two or more sensing devices and includes a categorization module and a functional performance recovery level module. The categorization module determines a latent category in response to the first signal, a manifested category in response to the second signal, and a significant category in response to both the first signal and the second signal. The functional performance recovery level module determines the patient's rehabilitative treatment progress in response to all of the latent category, the manifested category and the significant category.

According to another embodiment of the present invention, a non-transitory computer readable medium containing program instructions for causing a computer to perform a method for monitoring and determining progress of a patient's rehabilitative treatment is provided. The method includes determining a latent category in response to a segmented portion of a sensed physiological performance signal of a movement of a portion of a patient's body and determining a manifested category in response to a corresponding segmented portion of a sensed body portion movement signal of the movement of the portion of the patient's body. The method also includes determining a significant category in response to a dynamic functional connectivity measurement of the segmented portion of the sensed physiological performance signal of the movement of the portion of the patient's body and the corresponding segmented portion of the sensed body portion movement signal of the movement of the portion of the patient's body. Finally, the method includes determining the patient's rehabilitative treatment progress in response to all of the latent category, the manifested category and the significant category.

### BRIEF DESCRIPTION OF DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with a present embodiment.

FIG. 1 depicts a flowchart of operation of a system for monitoring and determining progress of a patient's rehabilitative treatment in accordance with a present embodiment.

FIG. 2 depicts an illustration of a system for monitoring and determining progress of a patient's rehabilitative treatment in accordance with the present embodiment.

FIG. 3 depicts a block diagram of the system for monitoring and determining progress of a patient's rehabilitative treatment of FIG. 2 in accordance with the present embodiment.

FIG. 4 depicts an illustration of a more detailed portion of the system for monitoring and determining progress of a patient's rehabilitative treatment of FIG. 3 in accordance with a present embodiment.

FIG. 5 depicts a block diagram of processing portions of the system of FIG. 3 in accordance with the present embodiment.

FIG. 6 depicts a block diagram illustrating the method for monitoring and determining progress of a patient's rehabilitative treatment in accordance with the present embodiment.

FIG. 7 depicts a flowchart of a sensing and segmentation algorithm of the method of FIG. 1 in accordance with the present embodiment.

FIG. 8 depicts a block diagram of significant category determination of the method of FIG. 1 in accordance with the present embodiment.

FIG. 9A depicts graphs of electromyography (EMG) and inertial measurement unit (IMU) sensed signals in accordance with the present embodiment, wherein FIG. 9A depicts EMG signals.

FIG. 9B depicts graphs of electromyography (EMG) and inertial measurement unit (IMU) sensed signals in accordance with the present embodiment, wherein FIG. 9B depicts IMU signals.

FIG. 10A depicts graphs and connectivity matrices for EMG and IMU signals for affected limbs of a rehabilitation patient in accordance with the present embodiment.

FIG. 10B depicts graphs and connectivity matrices for EMG and IMU signals for affected limbs of a rehabilitation patient in accordance with the present embodiment.

FIG. 10C depicts graphs and connectivity matrices for EMG and IMU signals for affected limbs of a rehabilitation patient in accordance with the present embodiment.

FIG. 10D depicts graphs and connectivity matrices for EMG and IMU signals for affected limbs of a rehabilitation patient in accordance with the present embodiment.

FIG. 10E depicts graphs and connectivity matrices for EMG and IMU signals for affected limbs of a rehabilitation patient in accordance with the present embodiment.

FIG. 10F depicts graphs and connectivity matrices for EMG and IMU signals for affected limbs of a rehabilitation patient in accordance with the present embodiment.

FIG. 10G depicts graphs and connectivity matrices for EMG and IMU signals for affected limbs of a rehabilitation patient in accordance with the present embodiment.

FIG. 10H depicts graphs and connectivity matrices for EMG and IMU signals for affected limbs of a rehabilitation patient in accordance with the present embodiment.

Skilled artisans will appreciate that elements in the figures are illustrated for simplicity and clarity and have not necessarily been depicted to scale.

### Description of Embodiments

The following detailed description is merely exemplary in nature and is not intended to limit the invention or the application and uses of the invention. Furthermore, there is no intention to be bound by any theory presented in the preceding background of the invention or the following detailed description. It is the intent of the present embodiment to present a method and a system for monitoring and determining progress of a patient's rehabilitative treatment which uses a combination of electromyography (EMG) and inertial measurement unit (IMU) signals to quantify the performance of the patient's functional abilities. The latent and manifested categories for the patient's functional abilities are determined from the EMG and the IMU signals separately while a significant category is quantitatively determined using functional connectivity between the EMG and the IMU signals, where such functional connectivity is missing for some patients who have limited ability of movement. Functional connectivity refers to a functionally integrated relationship between physiological performance represented by the EMG signals and range of motion of a body portion as represented by the IMU signals.

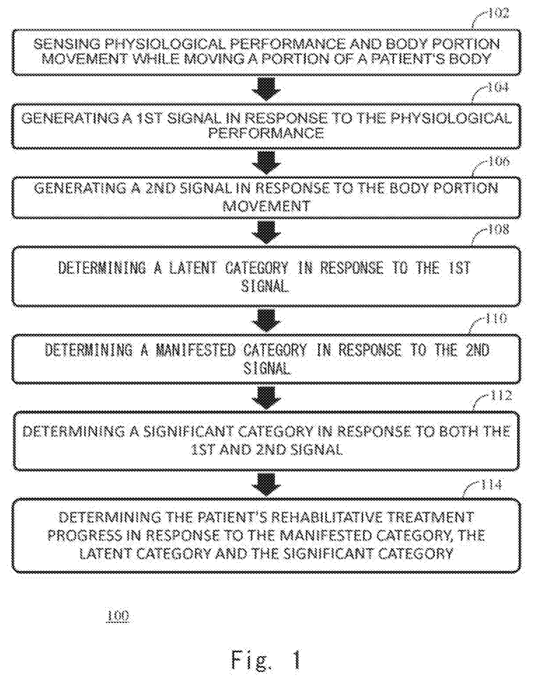

Referring to FIG. 1, a flowchart 100 depicts operation of a system for monitoring and determining progress of a patient's rehabilitative treatment in accordance with a present embodiment. Initially physiological performance and range of motion of a portion of a patient's body such as an arm, a leg, fingers or toes are sensed 102 while the patient moves that portion of his/her body. Then, a first signal is generated 104 in response to the sensed physiological performance of the portion of the patient's body. The physiological performance of the portion of the patient's body can be sensed by an EMG device or similar devices. Next, a second signal is generated 106 in response to the sensed body portion movement of the portion of the patient's body by, for example, an IMU device such as an accelerometer or a gyroscope.

Then, a latent category is determined 108 in response to the first signal and a manifested category is determined 110 in response to the second signal. In accordance with the present embodiment, a significant category 112 is determined in response to both the first signal and the second signal by determining a dynamic functional connectivity measurement of corresponding portions of the first signal and the second signal as discussed herein below. Finally, the patient's rehabilitative treatment progress is determined 114 by calculating an objective score in response to all of the latent category, the manifested category and the significant category.

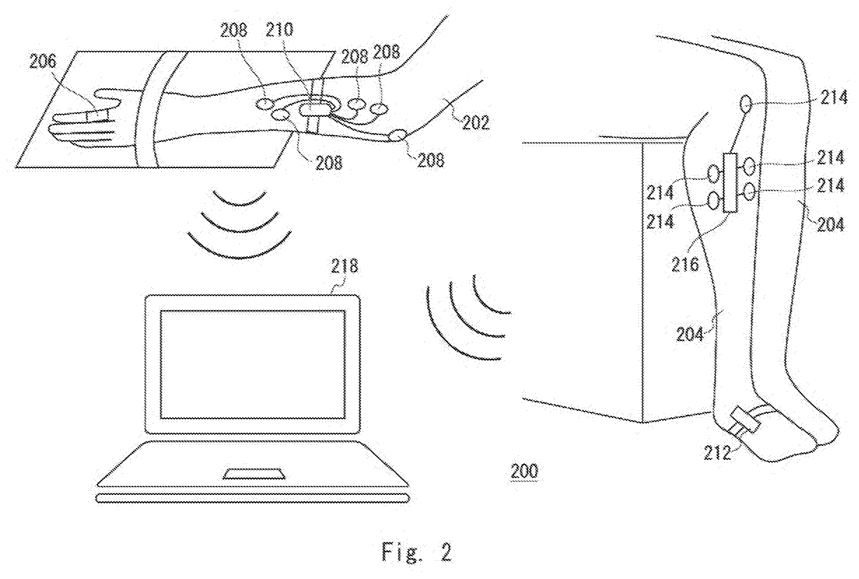

Referring to FIG. 2, an illustration 200 depicts monitoring and determining progress of a patient's rehabilitative treatment in accordance with the present embodiment. More particularly, the illustration 200 depicts physiological performance and range of motion sensing of a portion of a patient's body such as an arm 202 or a leg 204. An IMU device 206 on a finger measures the movement (i.e., range of motion) of the hand and electrodes 208 of a multichannel EMG device 210 detect physiological performance of the arm 202 while moving. Similarly, an IMU device 212 on a foot measures the movement (i.e., range of motion) of the foot and electrodes 214 of a multichannel EMG device 216 detect physiological performance of the leg 204 while moving. In accordance with the present embodiment, first signals from the EMG devices 210, 216 are transmitted to a processing device such as a laptop 218 and second signals from the IMU devices 206, 212 are transmitted to the laptop 218. In this manner, the first and second signals are generated in response to the physiological performance and the body portion movement, respectively, for processing by the laptop 218 in accordance with the present embodiment to monitor and determine a patient's progress during rehabilitative treatment.

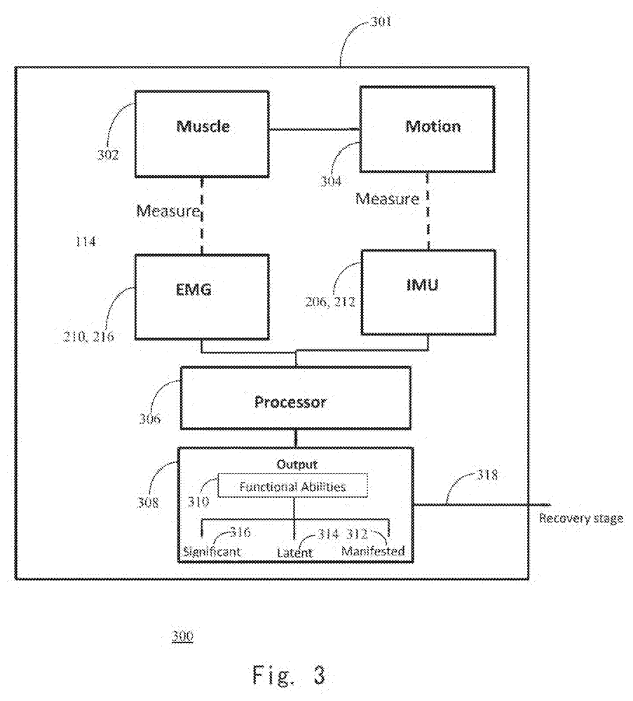

Referring to FIG. 3, a block diagram 300 of a system 301 for monitoring and determining progress of a patient's rehabilitative treatment in accordance with the present embodiment measures the physiological performance of a muscle 302 as the muscle 302 performs a specific motion 304. The multichannel EMG devices 210, 216 capture the muscle 302 activity and generate a first signal in response to that muscle 302 activity. The IMU devices 206, 212 capture the range of motion 304 and generate the second signal in response to that motion 304. The first signal and the second signal are provided to a processor 306 (e.g., a microprocessor of the laptop 218 (FIG. 2)) which extracts an output 308 by determining functional abilities 310.

The functional abilities 310 correspond to manifested category scores 312 determined in response to the second signal from the IMU devices 206, 212, latent category 314 determined in response to the first signal from the EMG devices 210, 216, and significant category 316 determined in response to the first and second signals. In accordance with the present embodiment, a recovery stage 318 can advantageously be determined from the output 308, such as an objective score of motor function. Thus, a more clinically relevant and stronger parameter for the prediction of functional assessment can be provided in accordance with the present embodiment by quantitatively determining a significant category from corresponding portions of both the first and second signals which expresses a dynamic functional connectivity between the physiological performance captured by the multichannel EMG devices 210, 216 and the range of motion captured by the IMU devices 206, 212.

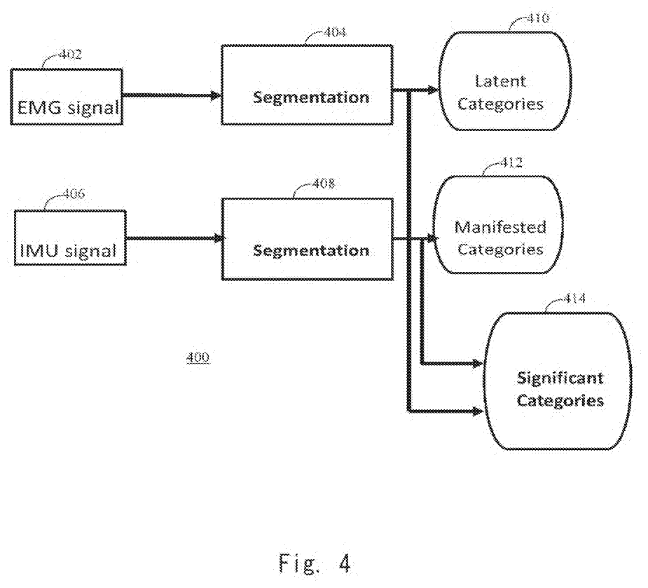

Referring to FIG. 4, an illustration 400 depicts how corresponding portions of the first and second signals are processed in accordance with a present embodiment. The first signal (i.e., the EMG signal 402) is provided to a first segmentation module 404 for segmenting the first signal into a plurality of segmented portions of the first signal. Likewise, the second signal (i.e., the IMU signal 406) is provided to a second segmentation module 408 for segmenting the second signal into a plurality of segmented portions of the second signal. The latent categories 410 are extracted from the segmented first signal so that a latent category can be determined in response to each of the plurality of segmented portions of the first signal. Likewise, the manifested categories 412 are extracted from the segmented second signal such that a manifested category can be determined in response to each of the plurality of segmented portions of the second signal. Finally, the significant categories 414 are determined in response to a dynamic functional connectivity measurement of corresponding segmented portions of the first signal and the second signal such that the significant category can be determined in response to each of the plurality of segmented portions of the first signal and a corresponding one of the plurality of segmented portions of the second signal.

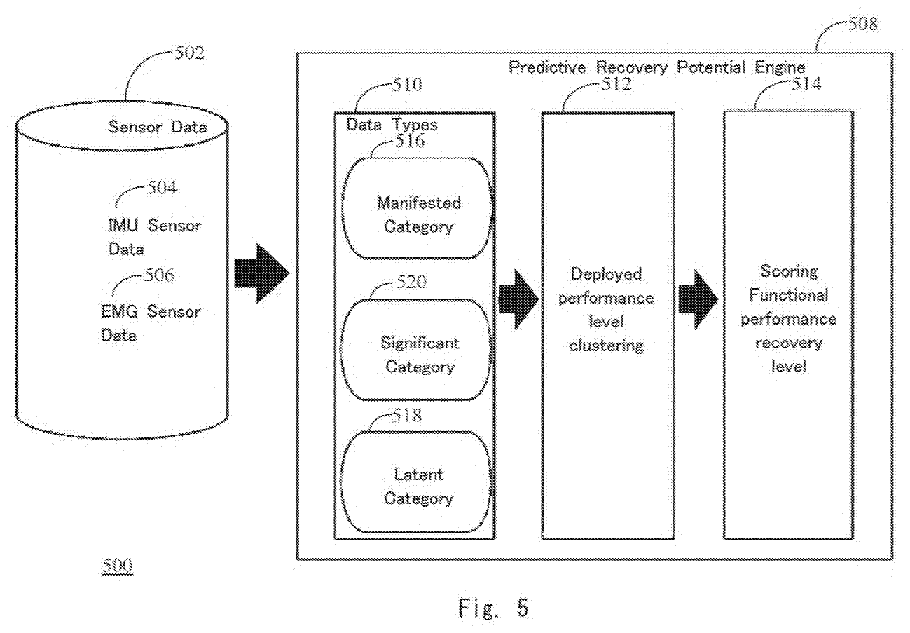

Referring to FIG. 5, a block diagram 500 depicts processing portions of system 301 (FIG. 3) in accordance with the present embodiment. Sensor data 502 stores IMU sensor data 504 and EMG sensor data 506 for later processing. In this manner, the patient's data can be obtained as first and second signals from two or more sensing devices while a portion of the patient's body (e.g., an arm or a leg) is moved and can be stored for later processing by a predictive recovery potential engine 508. The predictive recovery engine 508 includes a categorization module 510, a deployed performance level clustering module 512 and a scoring functional performance recovery level module 514. The categorization module 510 determines the manifested category 516 in response to the second signal (i.e., the IMU sensor data), determines a latent category 518 in response to the second signal (i.e., the EMG sensor data), and determines the significant category 520 in response to both the first signal and the second signal. By applying all three categories (manifested, latent and significant) in accordance with the present embodiment, the prediction of potential recovery of patients and planning of rehabilitative treatment strategies can be optimized.

The deployed performance level clustering module 512 determines a plurality of classifiers from the latent category 518, the manifested category516 and the significant category 520. The scoring functional performance recovery level module 514 determines an objective score representing the patient's rehabilitative treatment progress in response to the plurality of classifiers of the latent category 518, the manifested category 516 and the significant category 520.

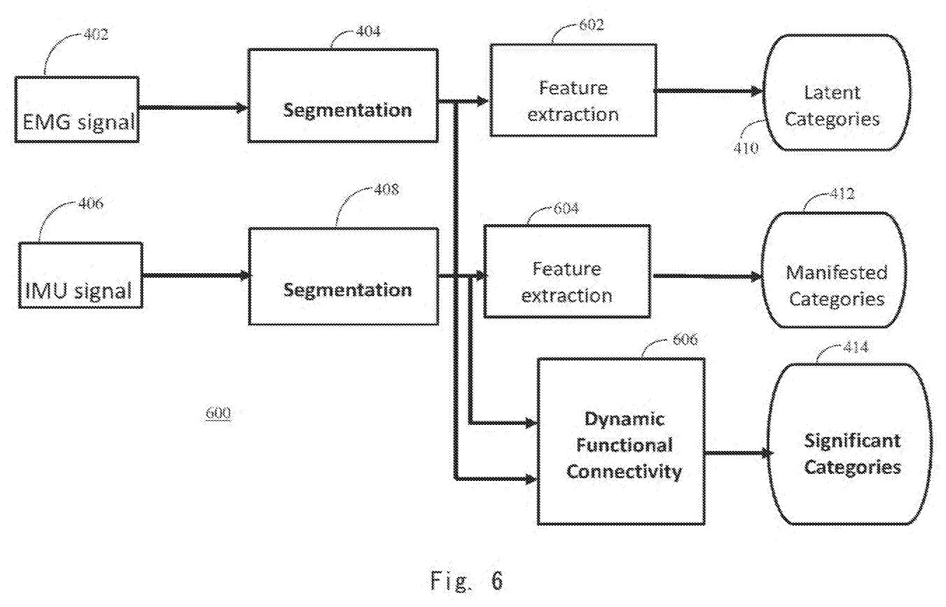

FIG. 6 depicts a block diagram 600 illustrating the method for monitoring and determining progress of a patient's rehabilitative treatment in accordance with the present embodiment. As described above, the acquired data includes the IMU signal 406 and the EMG signal 402. The IMU signal 406 can be acquired from one or more IMU sensors such as accelerometers or gyroscopes that can be used to determine a limb's orientation and/or acceleration, smoothness of motion and range of motion. The EMG signal can be acquired from a multichannel EMG device (e.g. EMG devices 210, 216) along with the IMU signal while the patient or subject performs a series of tasks. Advantageously, the tasks could be directed by visual and/or auditory instructions in accordance with the present embodiment, thereby removing the need for constant therapist or clinician monitoring and enabling improved correspondence between the EMG signal 402 and the IMU signal 406.

In accordance with the present embodiment, the EMG signal 402 can be pre-processed using baseline removal, band pass filtering and full wave rectification and smoothing. Most importantly, the EMG signal is normalized for example by using a maximum voluntary contraction normalization method. In addition, the IMU signal 406 is pre-processed. In accordance with the present embodiment, a complementary sensor fusion technology pre-processes the IMU signal 406 to accurately measure the elevation of movement of the patient or subject's limb.

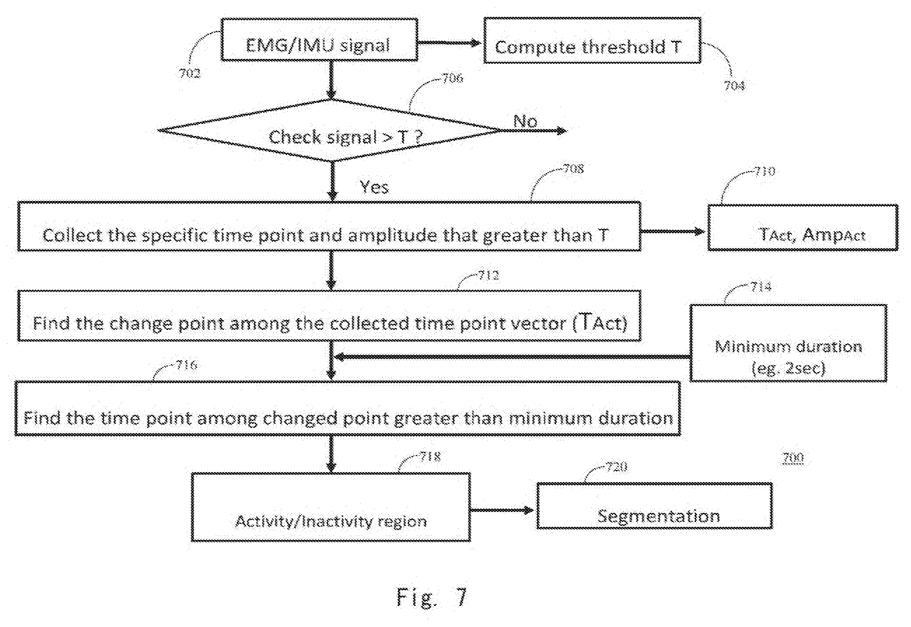

The segmentation modules 404, 408 automatically segment the EMG signal 402 and the IMU signal 406 based on a windowing method without overlapping as described in greater detail in FIG. 7. Feature extraction modules 602, 604 extract statistical features, time domain features and frequency domain features from the segmented portions of the EMG signal 402 and the IMU signal 406. For example, features such as peak amplitude, peak root-mean-square (RMS), peak frequency, mean frequency, median frequency and maximum power can be extracted by the feature extraction modules 602, 604 for each channel of the segmented portions of the EMG signal 402 and the IMU signal 406. For a patient who has high movement in a finger extension but weak or limited muscle activity, the manifested categories 412 determined from classification of the features extracted by the feature extraction module 604 will be higher than the latent categories 410 determined from classification of the features extracted by the feature extraction module 602. Conversely, for a patient who exhibits minimal movement in a finger extension but strong muscle activity, the manifested categories 412 determined from classification of the features extracted by the feature extraction module 604 will be lower than the latent categories 410 determined from classification of the features extracted by the feature extraction module 602.

In accordance with the present embodiment, the significant categories 414 are computed from integrating corresponding segmented portions of the EMG signal 402 and the IMU signal 406. Corresponding segmented portions means the segmented portions of the EMG signal 402 and the IMU signal 404 that fall within the same time window and therefore are EMG signals 402 and IMU signals 404 during the same motion of the limb or other body portion. A dynamic functional connectivity module 606 measures the functional correlation of corresponding segmented portions of the EMG signal 402 and the IMU signal 404 by defining a dynamic functional connectivity matrix between the EMG signals 402 and the IMU signals 404.

Referring to FIG. 7, a flowchart 700 depicts a sensing and segmentation algorithm in accordance with the present embodiment. A threshold based approach is an exemplary activity detection algorithm as shown in the flowchart 700. As the EMG signal 402 and/or the IMU signal 406 are received 702, initially a threshold T is computed 704 and then the value of the EMG signal 402 and/or the IMU signal 406 are compared with the threshold T to determine if the value is greater than T 706. The threshold T is computed 704 by combining the mean of the signal with the standard deviation of the signal.

When the threshold T is exceeded 706, a suspected activity is detected and the specific time point and the amplitude greater than T are collected 708. These are defined as TACT, and AmpACT 710. Then the difference between pairs of consecutive is determined 712 to find the change point among the collected time point vectors (TACT). Then, a minimum duration is defined 714 (e.g., two seconds) to find the activity/inactivity region in changed points. The time point among the changed points that are greater than the minimum duration 716 define when an activity starts and when an activity ends thereby defining the activity and inactivity regions 718. Thus, the EMG signal 402 and the IMU signal 406 are segmented 720 by the segmentation modules 404, 408 based on a windowing with overlapping method. Note that while two seconds is used as an example of a minimum duration, the minimum duration is not limited to this time period and can be changeable according to the characteristics of the signal.

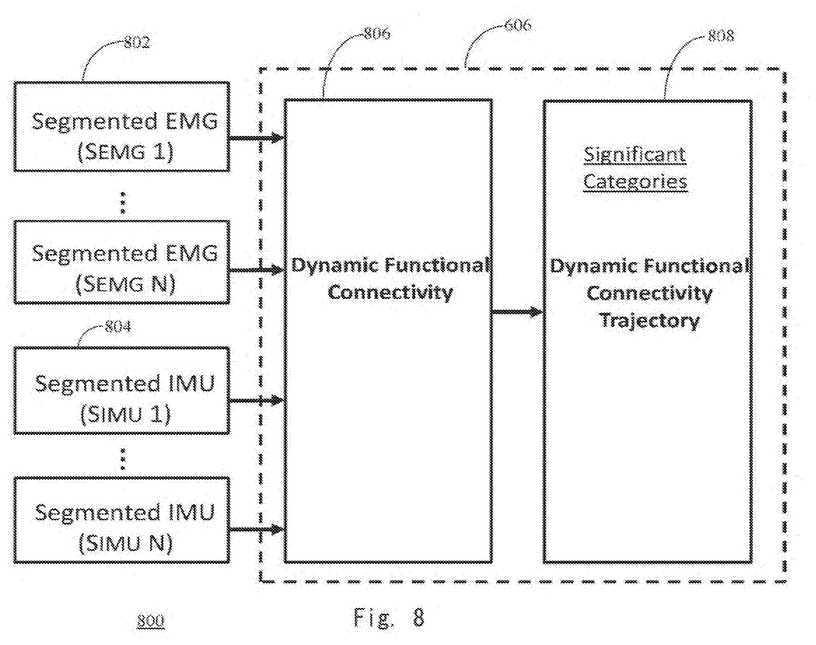

FIG. 8 depicts a block diagram 800 of significant category determination. The plurality of segmented EMG signal portions 802 and the plurality of segmented IMU signal portions 804 are provided to the dynamic functional connectivity module 606 to measure the functional correlation of corresponding segmented portions of the EMG signal 402 and the IMU signal 404. A dynamic functional connectivity unit 806 defines the dynamic functional connectivity matrix between the EMG signals 402 and the IMU signals 404. A dynamic functional connectivity trajectory unit 808 determines the significant categories by determining the dynamic functional connectivity trajectory of motor functional assessment of the limb or body portion being examined by averaging the functional connectivity matrix.

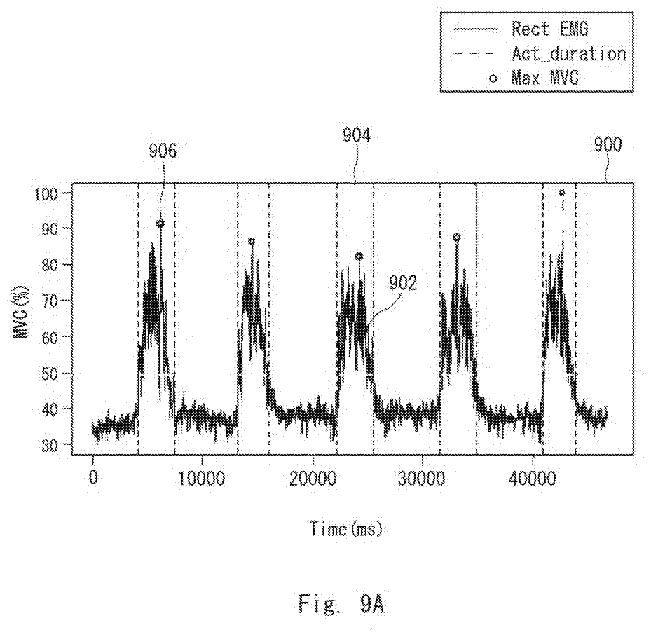

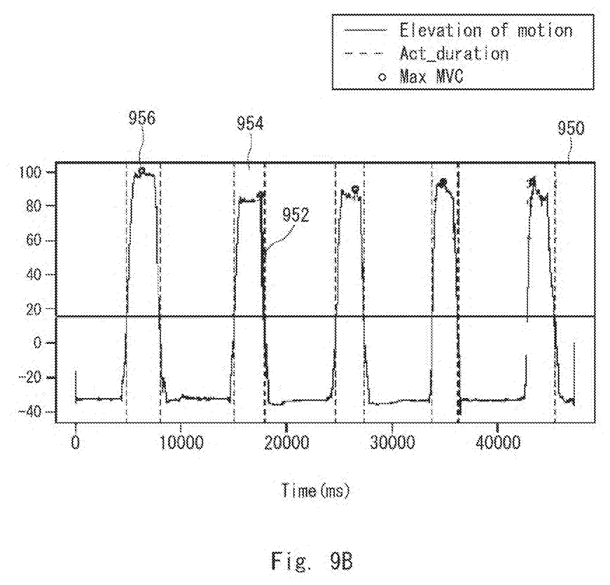

Referring to FIGS. 9A and 9B, graphs 900, 950 depict electromyography (EMG) and inertial measurement unit (IMU) sensed signals in accordance with the present embodiment. FIG. 9A depicts the graph 900 with EMG signals 902 segmented into active segments 904, a maximum EMG signal in each segment identified by points 906. FIG. 9B depicts the graph 950 with IMU signals 952 segmented into active segments 954, a maximum IMU signal in each segment identified by points 956.

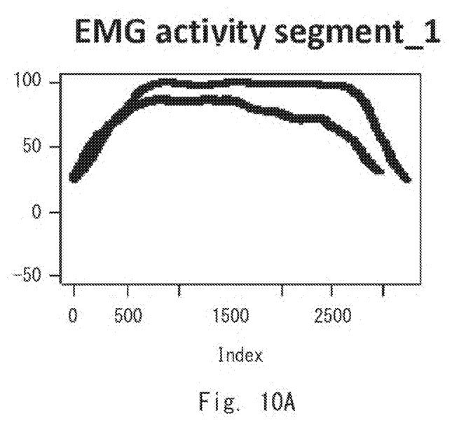

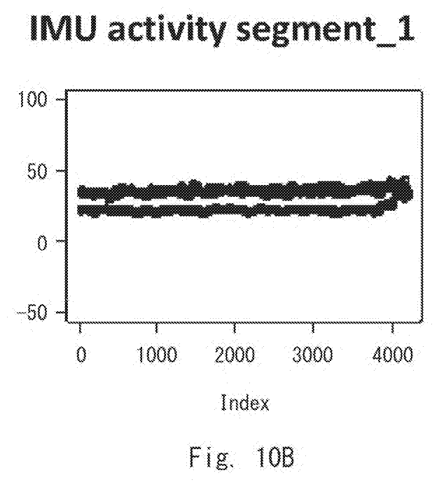

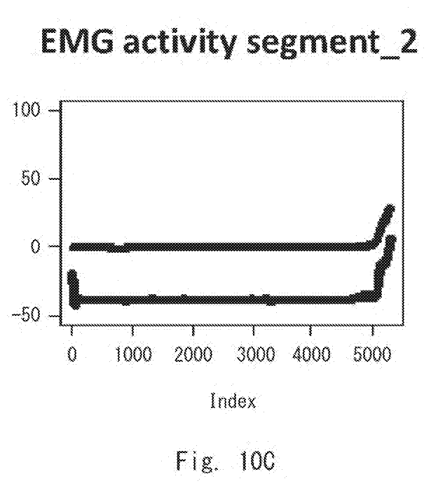

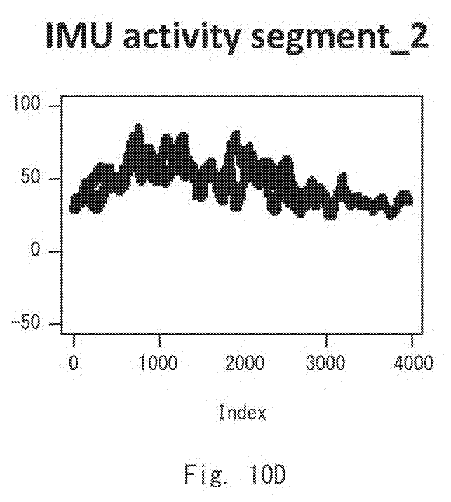

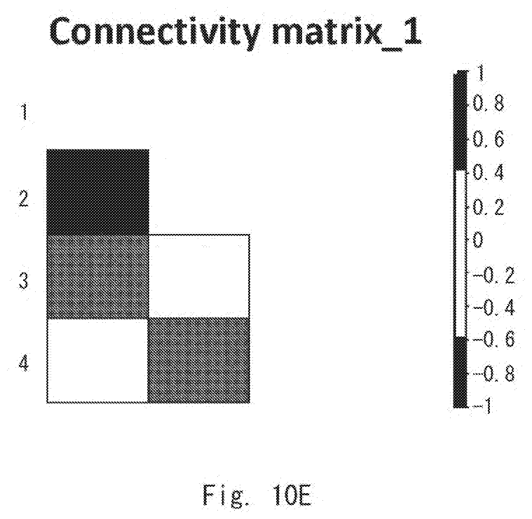

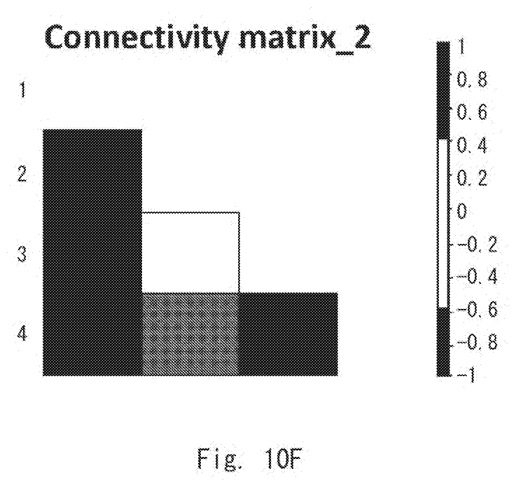

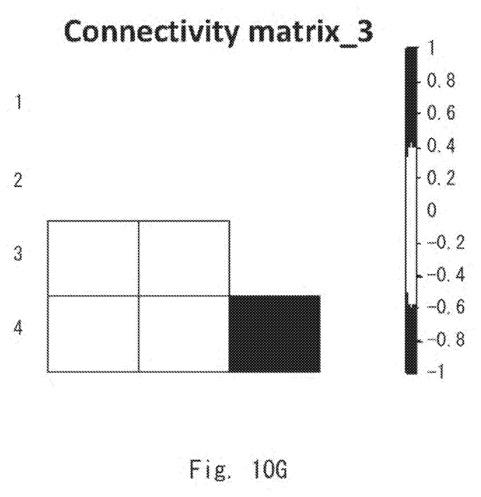

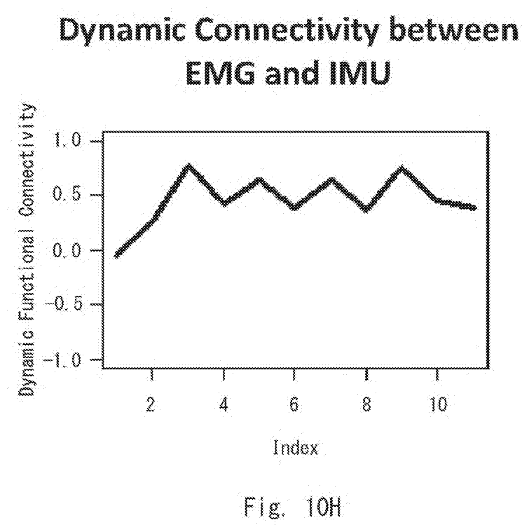

Referring to FIG. 10, graphs and connectivity matrices for EMG and IMU signals for affected limbs of a rehabilitation patient in accordance with the present embodiment are depicted. FIG. 10A depicts a graph of a first segment of EMG activity of an affected limb of the patient. FIG. 10B depicts a graph of a first segment of range of motion of an affected limb of the patient. FIG. 10C depicts a graph of a second segment of EMG activity of an affected limb of the patient. FIG. 10D depicts a graph of a second segment of range of motion of an affected limb of the patient. From these segmented regions of EMG and motion data, the connectivity matrices of IMU and EMG are computed for affected limb as shown in FIG. 10E (e.g., a first segmented connectivity matrix of the affected limb), FIG. 10F (e.g., a second segmented connectivity matrix of the affected limb) and FIG. 10G (e.g., a third segmented connectivity matrix of the affected limb). Finally, FIG. 10H depicts the dynamic functional connectivity of the affected limb from which the significant category is derived (see FIG. 6) by averaging the individual segmented connectivity matrices.

Thus, it can be seen that the present embodiment provides significant categories for functional connectivity to identify an individual's functional reorganization of neurologic recovery from brain injuries such as stroke. Disrupted functional connectivity will affect the performance of functional abilities. In accordance with the present embodiment, a system and method is provided to quantitatively determine significant categories, manifested categories and latent categories of a patient's functional abilities. Tuning parameters are provided in a detection algorithm in accordance with the present amendment for activities of EMG physiological data and IMU motion data and automatic segmentation.

While exemplary embodiments have been presented in the foregoing detailed description of the invention, it should be appreciated that a vast number of variations exist. It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of steps and method of operation described in the exemplary embodiment without departing from the scope of the invention as set forth in the appended claims.

The program can be stored and provided to the computer device using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (Read Only Memory), CD-R, CD-R/W, and semiconductor memories (such as mask ROM, PROM (Programmable ROM), EPROM (Erasable PROM), flash ROM, RAM (Random Access Memory), etc.). The program may be provided to the computer device using any type of transitory computer readable media. Examples of transitory computer readable media include electric signals, optical signals, and electromagnetic waves. Transitory computer readable media can provide the program to the computer device via a wired communication line, such as electric wires and optical fibers, or a wireless communication line.

For example, the whole or part of the exemplary embodiments disclosed above can be described as, but not limited to, the following supplementary notes.

(Supplementary Note 1)

A method for monitoring and determining progress of a patient's rehabilitative treatment, the method comprising:

sensing physiological performance and body portion movement while moving a portion of the patient's body;

generating a first signal in response to the sensed physiological performance of the portion of the patient's body;

generating a second signal in response to the sensed body portion movement of the portion of the patient's body;

determining a latent category in response to the first signal;

determining a manifested category in response to the second signal;

determining a significant category in response to both the first signal and the second signal; and

determining the patient's rehabilitative treatment progress in response to all of the manifested category, the latent category and the significant category.

(Supplementary Note 2)

The method in accordance with Supplementary note 1 wherein generating the first signal comprises:

generating a physiological performance signal in response to the sensed physiological performance of the portion of the patient's body; and

segmenting the physiological performance signal to generate the first signal.

(Supplementary Note 3)

The method in accordance with Supplementary note 1 or 2 wherein generating the second signal comprises:

generating a sensed movement signal in response to the sensed movement of the portion of the patient's body; and

segmenting the sensed movement signal to generate the second signal.

(Supplementary Note 4)

The method in accordance with Supplementary note 1 wherein generating the first signal comprises:

generating a physiological performance signal in response to the sensed physiological performance of the portion of the patient's body; and

segmenting the physiological performance signal to generate the first signal wherein the first signal comprises a plurality of segmented portions of the first signal, and wherein generating the second signal comprises:

generating a sensed movement signal in response to the sensed movement of the portion of the patient's body; and

segmenting the sensed movement signal to generate the second signal wherein the second signal comprises a plurality of segmented portions of the second signal, and

wherein determining the significant category comprises determining the significant category in response to a dynamic functional connectivity measurement of corresponding segmented portions of the first signal and the second signal.

(Supplementary Note 5)

The method in accordance with Supplementary note 1 wherein determining the latent category comprises:

extracting predetermined features [tuning parameters] from the first signal; and

determining the latent category in response to the predetermined features extracted from the first signal.

(Supplementary Note 6)

The method in accordance with Supplementary note 1 wherein determining the manifested category comprises:

extracting predetermined features from the second signal; and

determining the manifested category in response to the predetermined features extracted from the second signal.

(Supplementary Note 7)

The method in accordance with Supplementary note 1 wherein generating the first signal comprises generating the first signal in response to an electromyography (EMG) of the sensed physiological performance of the portion of the patient's body.

(Supplementary Note 8)

The method in accordance with Supplementary note 1 wherein generating the second signal comprises generating the second signal in response to inertial measurement units (IMU) of the sensed body portion movement of the portion of the patient's body.

(Supplementary Note 9)

The method in accordance with Supplementary note 1 wherein the portion of the patient's body moved comprises one of a patient's limb, a patient's hand, a patient's foot, a patient's fingers or a patient's toes.

(Supplementary Note 10)

A system for monitoring and determining progress of a patient's rehabilitative treatment, the system comprising:

two or more sensing devices comprising a first device for sensing physiological performance by the patient while moving a portion of the patient's body and generating a first signal in response thereto and a second device for sensing body portion movement by the patient while moving the portion of the patient's body and generating a second signal in response thereto; and

a predictive patient recovery potential module coupled to the two or more sensing devices and comprising:

a categorization module for determining a latent category in response to the first signal, determining a manifested category in response to the second signal, and determining a significant category in response to both the first signal and the second signal; and

a functional performance recovery level module for determining the patient's rehabilitative treatment progress in response to all of the latent category, the manifested category and the significant category.

(Supplementary Note 11)

The system in accordance with Supplementary note 10 wherein the categorization module comprises a first segmentation module for segmenting the first signal into a plurality of segmented portions of the first signal, the categorization module determining the latent category in response to each of the plurality of segmented portions of the first signal.

(Supplementary Note 12)

The system in accordance with Supplementary note 10 or Supplementary note 11 wherein the categorization module comprises a second segmentation module for segmenting the second signal into a plurality of segmented portions of the second signal, the categorization module determining the manifested category in response to each of the plurality of segmented portions of the second signal.

(Supplementary Note 13)

The system in accordance with Supplementary note 10 wherein the categorization module comprises a first segmentation module for segmenting the first signal into a plurality of segmented portions of the first signal and a second segmentation module for segmenting the second signal into a plurality of segmented portions of the second signal, and wherein the categorization module determines the latent category in response to each of the plurality of segmented portions of the first signal, determines the manifested category in response to each of the plurality of segmented portions of the second signal, and determines the significant category in response to a dynamic functional connectivity measurement of corresponding segmented portions of the first signal and the second signal.

(Supplementary Note 14)

The system in accordance with any of Supplementary note 10 to Supplementary note 13 wherein the categorization module comprises:

a first feature extraction module for extracting predetermined features from the first signal; and

a second feature extraction module for extracting predetermined features from the second signal, and

wherein the latent category is determined in response to the extracted predetermined features of the first signal, the manifested category is determined in response to extracted predetermined features of the second signal, and the significant category is determined in response to both the extracted predetermined features of the first signal and the extracted predetermined features of the second signal.

(Supplementary Note 15)

The system in accordance with any of Supplementary note 10 to Supplementary note 14 wherein the first device comprises an electromyography (EMG) device for sensing the physiological performance by the patient while moving the portion of the patient's body.

(Supplementary Note 16)

The system in accordance with any of Supplementary note 10 to Supplementary note 15 wherein the second device comprises an inertial measurement unit (IMU) device for sensing the body portion movement by the patient while moving the portion of the patient's body.

(Supplementary Note 17)

The system in accordance with any of Supplementary note 10 to Supplementary note 16 further comprising a deployed performance level clustering module coupled between the categorization module and the functional performance recovery level module for determining a plurality of classifiers from the latent category, the manifested category and the significant category, and wherein the functional performance recovery level module determines an objective score representing the patient's rehabilitative treatment progress in response to the plurality of classifiers.

(Supplementary Note 18)

The system in accordance with any of Supplementary note 10 to Supplementary note 17 wherein the portion of the patient's body moved comprises one of a patient's limb, a patient's hand, a patient's foot, a patient's fingers or a patient's toes.

(Supplementary Note 19)

A non-transitory computer readable medium containing program instructions for causing a computer to perform a method for monitoring and determining progress of a patient's rehabilitative treatment comprising:

determining a latent category in response to a segmented portion of a sensed physiological performance signal of a movement of a portion of a patient's body;

determining a manifested category in response to a corresponding segmented portion of a sensed body portion movement signal of the movement of the portion of the patient's body;

determining a significant category in response to a dynamic functional connectivity measurement of the segmented portion of the sensed physiological performance signal of the movement of the portion of the patient's body and the corresponding segmented portion of the sensed body portion movement signal of the movement of the portion of the patient's body; and

determining the patient's rehabilitative treatment progress in response to all of the latent category, the manifested category and the significant category.

This application is based upon and claims the benefit of priority from Singapore provisional patent application No. 10201800833T, filed on Jan. 31, 2018, the disclosure of which is incorporated herein in its entirety by reference.

### REFERENCE SIGNS LIST

206, 212 IMU device
208, 214 electrode
210, 216 EMG device
218 laptop
301 system
306 processor
308 output
402 EMG signal
406 IMU signal
404, 408 segmentation module
410 latent categories
412 manifested categories
414 significant categories
502 sensor data
510 categorization module
512 deployed performance level clustering module
514 functional performance recovery level module
602, 604 feature extraction module
606 dynamic functional connectivity module
802 segmented EMG signal portions
804 segmented IMU signal portions
806 dynamic functional connectivity unit
808 dynamic functional connectivity trajectory unit

What is claimed is:

1. A method for monitoring and determining progress of a patient's rehabilitative treatment, the method comprising:
sensing physiological performance and body portion movement while moving a portion of the patient's body;
generating a first signal in response to the sensed physiological performance of the portion of the patient's body;
generating a second signal in response to the sensed body portion movement of the portion of the patient's body;
determining a latent category in response to the first signal;
determining a manifested category in response to the second signal;
determining a significant category in response to both the first signal and the second signal; and
determining the patient's rehabilitative treatment progress in response to all of the manifested category, the latent category and the significant category.

2. The method in accordance with claim 1 wherein generating the first signal comprises:
generating a physiological performance signal in response to the sensed physiological performance of the portion of the patient's body; and
segmenting the physiological performance signal to generate the first signal.

3. The method in accordance with claim 1 wherein generating the second signal comprises:
generating a sensed movement signal in response to the sensed movement of the portion of the patient's body; and
segmenting the sensed movement signal to generate the second signal.

4. The method in accordance with claim 1 wherein generating the first signal comprises:
generating a physiological performance signal in response to the sensed physiological performance of the portion of the patient's body; and
segmenting the physiological performance signal to generate the first signal wherein the first signal comprises a plurality of segmented portions of the first signal, and wherein generating the second signal comprises:
generating a sensed movement signal in response to the sensed movement of the portion of the patient's body; and
segmenting the sensed movement signal to generate the second signal wherein the second signal comprises a plurality of segmented portions of the second signal, and
wherein determining the significant category comprises determining the significant category in response to a dynamic functional connectivity measurement of corresponding segmented portions of the first signal and the second signal.

5. The method in accordance with claim 1 wherein determining the latent category comprises:
extracting predetermined features [tuning parameters] from the first signal; and
determining the latent category in response to the predetermined features extracted from the first signal.

6. The method in accordance with claim 1 wherein determining the manifested category comprises:
extracting predetermined features from the second signal; and
determining the manifested category in response to the predetermined features extracted from the second signal.

7. The method in accordance with claim 1 wherein generating the first signal comprises generating the first signal in response to an electromyography (EMG) of the sensed physiological performance of the portion of the patient's body.

8. The method in accordance with claim 1 wherein generating the second signal comprises generating the second signal in response to inertial measurement units (IMU) of the sensed body portion movement of the portion of the patient's body.

9. The method in accordance with claim 1 wherein the portion of the patient's body moved comprises one of a patient's limb, a patient's hand, a patient's foot, a patient's fingers or a patient's toes.

10. A system for monitoring and determining progress of a patient's rehabilitative treatment, the system comprising:
two or more sensing devices comprising a first device for sensing physiological performance by the patient while moving a portion of the patient's body and generating a first signal in response thereto and a second device for sensing body portion movement by the patient while moving the portion of the patient's body and generating a second signal in response thereto; and
a predictive patient recovery potential module coupled to the two or more sensing devices and comprising:
a categorization module for determining a latent category in response to the first signal, determining a manifested category in response to the second signal, and determining a significant category in response to both the first signal and the second signal; and
a functional performance recovery level module for determining the patient's rehabilitative treatment progress in response to all of the latent category, the manifested category and the significant category.

11. The system in accordance with claim 10 wherein the categorization module comprises a first segmentation module for segmenting the first signal into a plurality of segmented portions of the first signal, the categorization module determining the latent category in response to each of the plurality of segmented portions of the first signal.

12. The system in accordance with claim 10 wherein the categorization module comprises a second segmentation module for segmenting the second signal into a plurality of segmented portions of the second signal, the categorization module determining the manifested category in response to each of the plurality of segmented portions of the second signal.

13. The system in accordance with claim 10 wherein the categorization module comprises a first segmentation module for segmenting the first signal into a plurality of segmented portions of the first signal and a second segmentation module for segmenting the second signal into a plurality of segmented portions of the second signal, and wherein the categorization module determines the latent category in response to each of the plurality of segmented portions of the first signal, determines the manifested category in response to each of the plurality of segmented portions of the second signal, and determines the significant category in response to a dynamic functional connectivity measurement of corresponding segmented portions of the first signal and the second signal.

14. The system in accordance with claim 10 wherein the categorization module comprises:
a first feature extraction module for extracting predetermined features from the first signal; and
a second feature extraction module for extracting predetermined features from the second signal, and
wherein the latent category is determined in response to the extracted predetermined features of the first signal, the manifested category is determined in response to extracted predetermined features of the second signal, and the significant category is determined in response to both the extracted predetermined features of the first signal and the extracted predetermined features of the second signal.

15. The system in accordance with claim 10 wherein the first device comprises an electromyography (EMG) device for sensing the physiological performance by the patient while moving the portion of the patient's body.

16. The system in accordance with claim 10 wherein the second device comprises an inertial measurement unit (IMU) device for sensing the body portion movement by the patient while moving the portion of the patient's body.

17. The system in accordance with claim 10 further comprising a deployed performance level clustering module coupled between the categorization module and the functional performance recovery level module for determining a plurality of classifiers from the latent category, the manifested category and the significant category, and wherein the functional performance recovery level module determines an objective score representing the patient's rehabilitative treatment progress in response to the plurality of classifiers.

18. The system in accordance with claim 10 wherein the portion of the patient's body moved comprises one of a patient's limb, a patient's hand, a patient's foot, a patient's fingers or a patient's toes.

19. A non-transitory computer readable medium containing program instructions for causing a computer to perform a method for monitoring and determining progress of a patient's rehabilitative treatment comprising:
determining a latent category in response to a segmented portion of a sensed physiological performance signal of a movement of a portion of a patient's body;
determining a manifested category in response to a corresponding segmented portion of a sensed body portion movement signal of the movement of the portion of the patient's body;
determining a significant category in response to a dynamic functional connectivity measurement of the segmented portion of the sensed physiological performance signal of the movement of the portion of the patient's body and the corresponding segmented portion of the sensed body portion movement signal of the movement of the portion of the patient's body; and
determining the patient's rehabilitative treatment progress in response to all of the latent category, the manifested category and the significant category.
